(19) United States
(12) Patent Application Publication
(10) Pub. No.: US 2008/0108899 A1
(43) Pub. Date: May 8, 2008

(54) Hand-held ultrasound system with single integrated circuit back-end

(76) Inventors: Nahi Halmann, Milwaukee, WI (US); Zhenhai Lou, Wuxi (CN); Kai Ji, Wuxi (CN)

Correspondence Address: DEAN D. SMALL;THE SMALL PATENT LAW GROUP LLP, 611 OLIVE STREET, SUITE 1611, ST. LOUIS, MO, 63101, US

(21) Appl. No.: 11/593,243

(22) Filed: Nov. 6, 2006

(51) Int. Cl. A61B 8/00

(52) U.S. Cl. 600/459

(57) ABSTRACT

An ultrasound system comprises a front-end and a back-end. The front-end acquires ultrasound data indicative of a subject and comprises a probe, transmitter, receiver and beamformer. The probe has a plurality of transducer elements which are driven by the transmitter to transmit ultrasonic signals into the subject. The receiver detects returned echoes based on the ultrasonic signals, and the beamformer receives the returned echoes from the receiver and outputs a beamformed signal. The back-end comprises a single integrated circuit (IC). The back-end receives the beamformed signal from the front-end. The back-end processes the beamformed signal and outputs ultrasound image data based on the beamformed signal.

### BACKGROUND OF THE INVENTION

This invention relates generally to ultrasound systems, and more particularly, to small-size ultrasound systems.

The majority of ultrasound systems in use today are relatively large, being cart-based and/or wheel-carried. The systems carry with them one or more probes for different types of scanning procedures. The systems may be based on a personal computer (PC) platform and also have multiple circuit boards. The systems are not hand-held or hand-carried in that the systems cannot be easily picked up and moved by a single person, but are instead wheeled, rolled, transported via elevator to a different floor, or by a special vehicle if moving to a different physical location. Therefore, the physical constraints of moving the ultrasound system limits its portability, as well as the amount of space needed during operation of the system.

Physicians desire the use of ultrasound systems for some surgical procedures in the operating room or when performing ultrasound guided procedures. Unfortunately, the size of the ultrasound system may limit or prohibit its use in the operating room, where space is at a premium.

Also of concern is the power consumption, which can be great in a higher end ultrasound machine. As more power is consumed, more heat is dissipated which increases the cooling requirements of the room the system is being used in, as well as requiring hardware such as fans to move ambient air through the system to cool components. Also, more battery power is needed to operate the system, and/or the system may operate for less time when relying on battery power.

Therefore, a need exists to miniaturize both the size and power consumption of ultrasound machines to provide additional flexibility and portability. Certain embodiments of the present invention are intended to meet these needs and other objectives that will become apparent from the description and drawings set forth below.

### BRIEF DESCRIPTION OF THE INVENTION

In one embodiment, an ultrasound system comprises a front-end and a back-end. The front-end acquires ultrasound data indicative of a subject and comprises a probe, transmitter, receiver and beamformer. The probe has a plurality of transducer elements which are driven by the transmitter to transmit ultrasonic signals into the subject. The receiver detects returned echoes based on the ultrasonic signals, and the beamformer receives the returned echoes from the receiver and outputs a beamformed signal. The back-end comprises a single integrated circuit (IC). The back-end receives the beamformed signal from the front-end. The back-end processes the beamformed signal and outputs ultrasound image data based on the beamformed signal.

In another embodiment, a diagnostic medical imaging system comprises a front-end portion, a back-end portion and a housing. The front-end portion acquires imaging data indicative of a patient. The back-end portion comprises at least one multiple-core IC that has at least first and second cores. The first core runs an operating system and at least one user interface, and the second core performs signal and image processing. The housing holds the front-end portion and the back-end portion there-within.

In another embodiment, a method for acquiring and processing ultrasound data comprises acquiring ultrasound data using a front-end of an ultrasound system. An operating system (OS) and user interface are run with a first core of a single IC that is based on a multiple-core architecture comprising at least first and second cores. Signal and image processing are performed with the second core of the single IC.

### BRIEF DESCRIPTION OF THE DRAWINGS

FIG. 1 illustrates a block diagram of an ultrasound system that uses components to facilitate both a smaller size system and less power consumption formed in accordance with an embodiment of the present invention.

FIG. 2 illustrates a conceptual block diagram of the software architecture of the back-end of FIG. 1 which is implemented on a single dual-core integrated circuit (IC) in accordance with an embodiment of the present invention.

FIG. 3 illustrates a conceptual block diagram of the software architecture of the back-end of FIG. 1 being implemented on a single multiple-core IC in accordance with an embodiment of the present invention.

FIG. 4 illustrates a block diagram of components within the system of FIG. 1 in accordance with an embodiment of the present invention.

FIG. 5 illustrates a hand-held ultrasound system formed in accordance with an embodiment of the present invention.

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

### DETAILED DESCRIPTION OF THE INVENTION

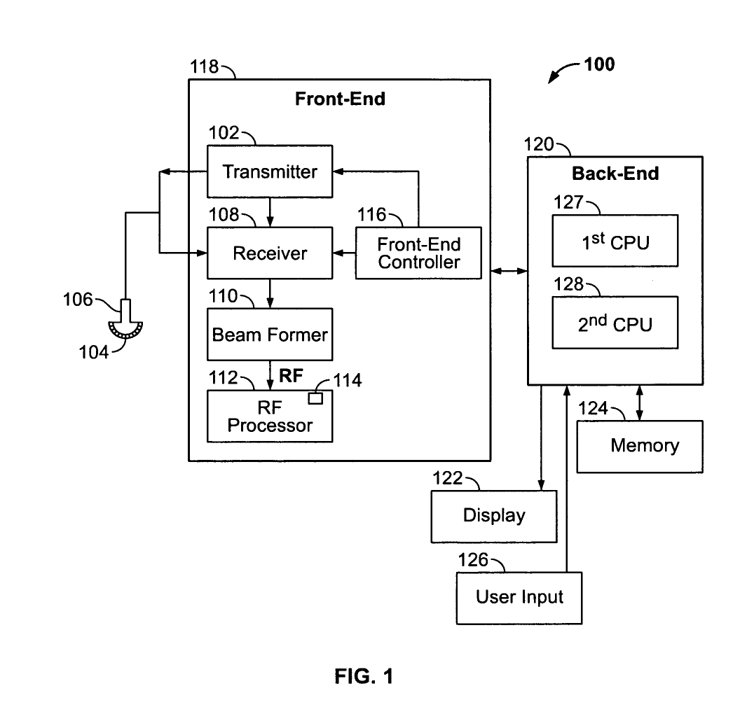

FIG. 1 illustrates a block diagram of an ultrasound system 100 that uses components to facilitate both a smaller size system 100 and less power consumption. By way of example, the system 100 may be hand-held, hand-carried, or pocket-sized. Alternatively, the system 100 may be cart-based while being smaller, more lightweight and portable, and consuming less overall power compared to conventional cart-based ultrasound systems. Alternatively, the system 100 may interface with other cart-based components, such as a stand-alone monitor and a keyboard (not shown).

The ultrasound system 100 includes a probe 106, a front-end 118, and a back-end 120. The front-end 118 generally refers to the electronic circuitry that handles the transmit and receive beam forming, as well as the real-time control of the probe 106. The front-end 118 is typically implemented by hardware, using one or more circuit boards.

Previously, the back-end 120 was implemented using multiple circuit boards and other hardware components which require a large amount of power and space. In system 100, however, a single chip or integrated circuit (IC) performs the back-end functionality. By using the single IC, the system 100 may be much smaller than a conventional ultrasound system, providing the user more flexibility in location of use and portability, as well as realizing a lower cost. The single IC may be a dual-core architecture or other multiple-core architecture as discussed below, and may also be referred to a system-on-chip (SoC) platform. Dual-core and multiple-core processors have multiple independent processor cores integrated on a single chip.

The back-end 120 receives a beamformed signal from the front-end 118 and implements the image processing, display functionality to display text and image data, and handles user interface events. The back-end 120 also provides additional software functionality, such as performing measurements on images, annotations, archiving, reporting, printing images, networking, and the like.

By way of example, implementing the back-end 120 by using a single IC may consume less than 2 Watts of power. The system 100 may thus consume under 5 Watts or 10 Watts of total system power, including the power needed for operation of the probe 106. In contrast, a typical ultrasound system, which may use a laptop computer or other small personal computer for processing, consumes 15-20 watts of power. The power is dissipated as heat which needs to be moved outside of the ultrasound system to protect components from damage. Therefore, using the single IC greatly reduces thermal dissipation in addition to lowering the power consumption.

As illustrated, the back-end 120 comprises a dual-core technology having first and second CPUs 127 and 128. The first CPU 127 may run an operating system (OS) and the second CPU 128 may support digital signal processing. Multiple-core technologies, such as four or eight cores, provide multiple CPUs which may support different functionality within each core, or may provide more than one core supporting digital signal processing.

Within the front-end 118, a transmitter 102 drives transducer elements 104 within the probe 106 to emit pulsed ultrasonic signals into a body. A variety of geometries may be used. The ultrasonic signals are back-scattered from structures in the body, like blood cells or muscular tissue, to produce echoes that return to the transducer elements 104. The echoes are received by a receiver 108. The received echoes are passed through a beamformer 110, which performs beamforming and outputs an RF signal. The RF signal then passes through an RF processor 112 which may include a complex demodulator 114 that demodulates the RF signal to form IQ data pairs representative of the echo signals. The RF processor may also detect and compress the signal, to further reduce its bandwidth. The output of the RF processor 112 may also be referred to as a beamformed signal. A front-end controller 116 controls the transmitter 102 and the receiver 108.

The back-end 120 processes the acquired ultrasound information (i.e., beamformed signal, RF signal data or IQ data pairs) and prepares frames of ultrasound information for display on display 122. One or more processing operations may be performed according to a plurality of selectable ultrasound modalities on the acquired ultrasound information. Acquired ultrasound imaging data may be processed in real-time during a scanning session as the echo signals are received. Additionally or alternatively, the ultrasound information may be stored in a memory 124 during a scanning session and processed in less than real-time in a live or off-line operation.

The memory 124 may comprise any known data storage medium, may be provided integral with, in addition to, or separable from, the system 100. For example, the memory 124 may be a hard drive, CD Rom, DVD, flash memory, memory stick, or any other memory or memory device.

A user input 126 may be used to control operation of the ultrasound system 100, including, for example, inputting patient data and scan parameters, changing a scanning mode, and the like. A microphone (not shown) may be used to input voice commands. The user input 126 may provide input capability through a keyboard, a touch screen or panel, switches, buttons, and the like.

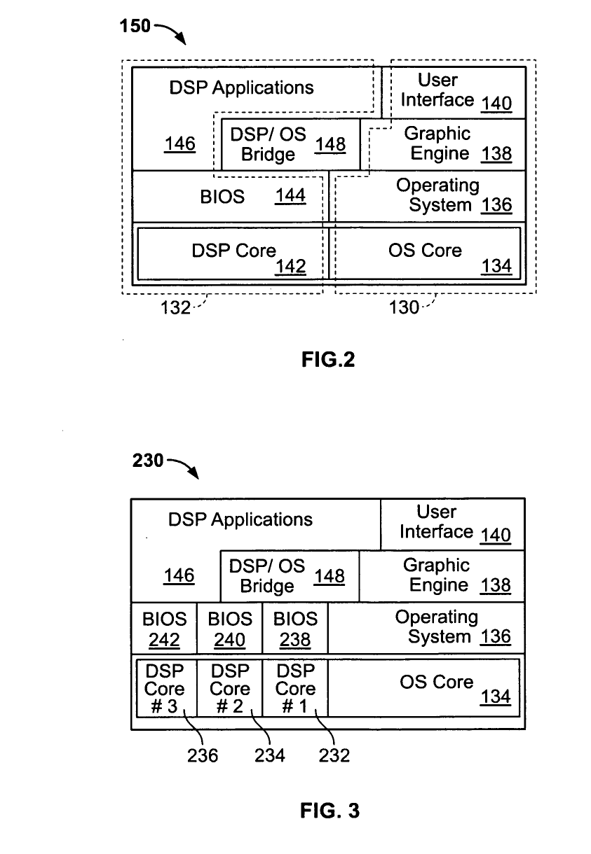

FIG. 2 illustrates a conceptual block diagram of the software architecture of the back-end 120 of FIG. 1, which is implemented on a single dual-core IC 150. Operating system (OS) core 134 (corresponding to the first CPU 127 FIG. 1) may be a general purpose CPU running a standard operating system such as Windows or Linux, for example. The OS core 134 implements the user interface functionality, as well as performing measurements on images, annotations, archiving, reporting, printing images, networking, and the like. Digital Signal Processor (DSP) core 142 (corresponding to the second CPU 128 of FIG. 1) is a DSP that performs the real-time image processing tasks needed to generate an image. The image processing tasks may include temporal frame averaging, CFM processing, Doppler processing, B mode and CFM scan conversion (conversion of the acoustic raw data to screen coordinates) and display. The OS and DSP cores 134 and 142 use shared resources, such as one or more shared memories (not shown), which may typically be allocated at boot time.

The dual-core IC 150 is conceptually divided primarily into first and second portions 130 and 132. The first portion 130 comprises the OS core 134, OS 136, graphic engine 138, and user interface 140. The second portion 132 comprises the DSP core 142, BIOS 144, and DSP applications 146. DSP/OS bridge 148 indicates a software interconnection between the first and second portions 130 and 132, which may also be referred to as interprocess communication (IPC). When programming the dual-core IC 150, the DSP applications 146 may be downloaded as a first customized program while the graphic engine 138 and user interface 140 may be downloaded as a second customized program. The first and second customized programs, as well as additional software programs which may be needed, may be loaded into, for example, a flash memory (not shown) or other memory.

FIG. 3 illustrates a conceptual block diagram of the software architecture of the back-end 120 being implemented on a single multiple-core IC 230. The multiple-core IC 230 may have four different cores as illustrated. The OS core 134, OS 136, graphic engine 138 and user interface 140 may be similar to the link-numbered items as discussed in FIG. 2. The multiple-core IC 230 may have first, second and third DSP cores 232, 234, and 236 having first, second, and third BIOS 238, 240, and 242, respectively. The DSP/OS bridge 148 indicates the software interconnection between the OS core 134, the first DSP core 232, second DSP core 234, and third DSP core 236.

The multiple-core IC 230, having three DSP cores, may provide greater processing power when compared to the single DSP core 142 of the dual-core IC 150. Optionally, the additional DSP cores may enable the multiple-core IC 230 to replace other hardware components within the system 100, allowing additional miniaturization. Optionally, additional dual-core ICs and/or multiple-core ICs may be used to increase processing speed and/or further miniaturize the system 100.

Optionally, one or more applications may be configured to migrate dynamically from one core to another based on current utilization of each core. This migration may find more application with ICs having four or eight cores than with ICs having two cores. Optionally, application threads may be executed according to priority by the first available CPU core. Optionally, software tasks may be locked to specific cores by using bound multiprocessing.

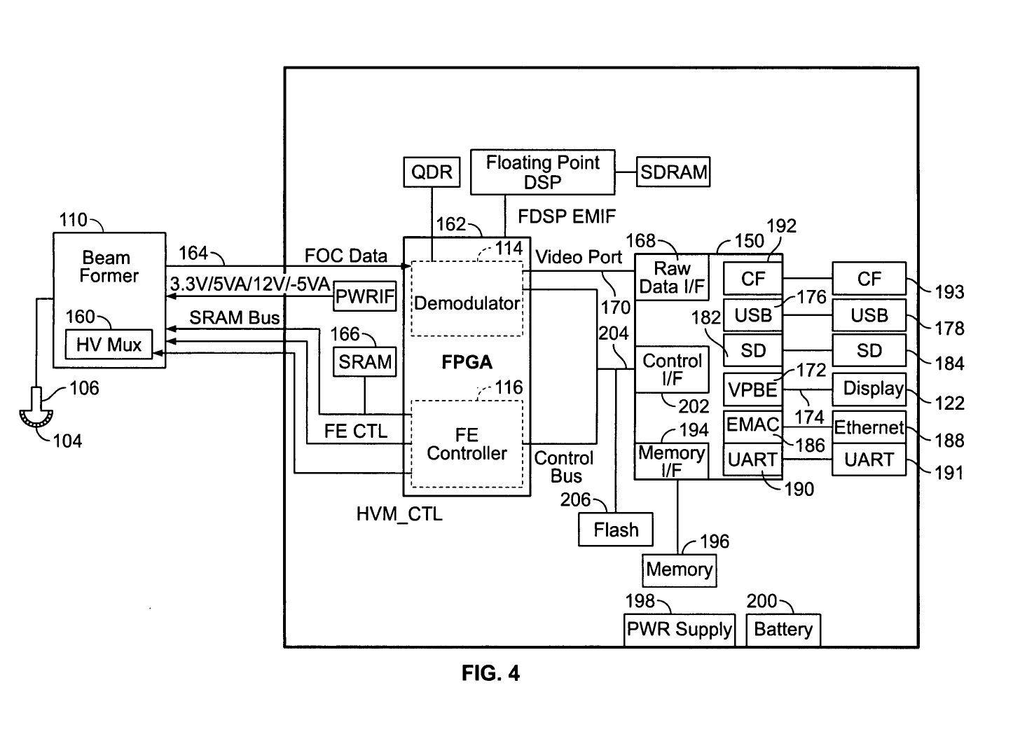

FIG. 4 illustrates a block diagram of components within the system 100. A high voltage multiplexer (HV MUX) 160 may be integral with or separate from the beamformer 110. The HV MUX 160 may be used to switch between the transducer elements 104 of the probe 106 when the system 100 has less channels, such as when the probe 106 has 128 or 265 transducer elements 104, while the system 100 has 64 channels.

A single IC, such as a field programmable gate array (FPGA) 162, may be used to accomplish the demodulator 114 and front-end controller 116 operations. An SRAM memory 166 may be an external memory provided to support the FPGA 162. The FPGA 162 is a programmable device that handles all real-time front-end control, such as setting up the beamformer 110. Therefore, the FPGA 162 handles all of the digital control and signal processing that is still accomplished in hardware, versus the functionality provided in software by the dual-core IC 150.

The demodulator 114 accepts incoming data from the beamformer 110 on line 164. The demodulator 114 reduces the data to the baseband of the acoustic information, which is the basic signal processing accomplished in hardware.

A plurality of communication ports or interfaces is available on the dual-core IC 150. Raw data interface (I/F) 168 accepts real-time video data from the demodulator 114 (the hardware side) via line 170. The raw ultrasound data is coming into the second portion 132 (FIG. 2) of the dual-core IC 150 for signal processing and display.

Control interface (I/F) port 202 provides an interface to acquire and send control data to and from the demodulator 114 and the front-end controller 116 on line 204. Flash memory 206 may store the software for the OS and DSP cores 134 and 142 (FIG. 2), which is then loaded into RAM memory (not shown) during execution.

The dual-core IC 150 also interfaces to the display 122 via video port back-end interface (VPBE) 172 and line 174. The dual-core IC 150 may provide one or more additional communication interfaces. For example, a USB port 176 may accept a USB device 178, such as a memory stick, or a USB cable. A secure digital (SD) port 182 (or mini-SD) may accept an SD device 184, an Ethernet (EMAC) port 186 may accept an Ethernet cable 188 or device, a UART port 190 may accept a UART cable 191 or device, and a compact flash (CF) port 192 may accept a CF device 193. Memory interface (I/F) 194 is provided to interface with memory 196, which may be an SDRAM memory controller. It should be understood that other ports and/or interfaces may be used. By way of example, image storage and software upgrades may be accomplished through one or more of the communication interfaces.

A power supply 198 may receive power from an external electrical source (not shown) and, when external power is not available, from a battery 200. Therefore, the system 100 may operate under either external power or battery power. The power supply 198 may supply a plurality of different voltage levels to meet the different operational needs of the components within the system 100. The external power source may also be used to keep the battery 200 in a charged condition.

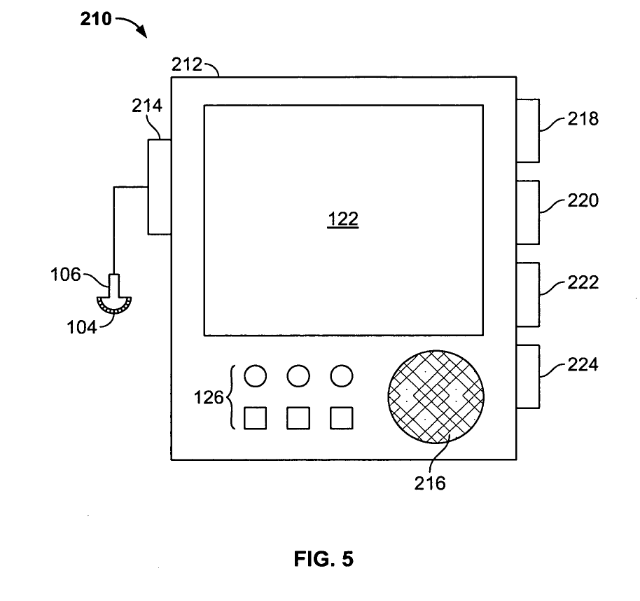

FIG. 5 illustrates a hand-held ultrasound system 210. Except for the probe 106, the components and functionality previously discussed in FIGS. 1-4 may be held within a single housing 212, which is small in size. For example, the system 210 may be less than 500 grams, less than 1 KG, or less than 3 Kg in weight. Additionally, the system 210 may be small in size to be easily held in an operator's hand and/or carried within an operator's pocket.

The housing 212 may have the display 122 and the user input 126 as previously discussed. A probe interconnection port 214 allows different probes 106 to be connected to the system 210 via the housing 212. A speaker 216 may be provided for outputting sound.

Optionally, additional ports may be provided to support other peripheral components. For example, an operator may wish to use the system 210 as a cart-based system for some applications. A cable (not shown) may interconnect video output port 218 to an external monitor (not shown) which may be larger than the display 122. Also, a keyboard input port 220 may allow an external full-size keyboard (not shown) to input data into the system 210. Additional ports 222 and 224 may be provided to allow the use of additional peripherals, such as an ECG, printer, and the like. In addition, ports (not shown) corresponding to the output ports discussed in FIG. 4, such as VPBE 172, USB port 176, SD port 182, EMAC port 186, UART port 190, and CF port 192, may be provided.

Although the dual-core IC 150 (FIG. 2) and multiple-core IC 230 (FIG. 3) as discussed have been implemented within a hand-held ultrasound system, it should be understood that the technology is applicable to cart-based ultrasound systems as well as to other medical modalities. Functionality previously accomplished by multiple hardware components, as was accomplished by the back-end used in prior ultrasound machines, may be accomplished by replacing some of the components with one or more dual-core and/or multiple-core ICs which consume less power, dissipate less heat, and have a much smaller footprint. This may further allow for additional modularity within medical systems, allowing a system to be portable as well as interconnected with cart-based components.

A technical effect is the ability to operate a medical diagnostic system while requiring less space and less power. The hand-held ultrasound system which incorporates one or more dual-core ICs and/or multiple-core ICs to accomplish the functionality of the back-end is much smaller than previous cart-based ultrasound systems. In addition to a smaller footprint, the hand-held ultrasound system is light in weight, providing an operator the flexibility of easily carrying and/or transporting the system.

While the invention has been described in terms of various specific embodiments, those skilled in the art will recognize that the invention can be practiced with modification within the spirit and scope of the claims.

What is claimed is:

1. An ultrasound system, comprising:
a front-end for acquiring ultrasound data indicative of a subject, the front-end further comprising:
a probe comprising a plurality of transducer elements;
a transmitter driving the plurality of transducer elements to transmit ultrasonic signals into the subject;
a receiver detecting returned echoes based on the ultrasonic signals;
a beamformer receiving the returned echoes from the receiver and outputting a beamformed signal; and
a back-end receiving the beamformed signal from the front-end, the back-end processing the beamformed signal and outputting ultrasound image data based on the beamformed signal, the back-end comprising a single integrated circuit (IC).

2. The ultrasound system of claim 1, wherein the single IC is based on one of a dual-core architecture and a multiple-core architecture, the multiple-core architecture having more than two cores.

3. The ultrasound system of claim 1, wherein the system consumes one of under 5 Watts of total system power and under 10 Watts of total system power.

4. The ultrasound system of claim 1, wherein the single IC provides functionality for at least one of real-time control of the front-end, user interface, scan conversion, CFM processing, Doppler processing, B mode scan conversion, CFM scan conversion, temporal frame processing, color processing, Doppler processing, and display processing.

5. The ultrasound system of claim 1, wherein the single IC is based on a dual-core architecture comprising first and second cores, the first core comprising a general purpose CPU and the second core comprising a programmable digital signal processor.

6. The ultrasound system of claim 1, wherein the single IC is based on a dual-core architecture comprising an operating system (OS) core and a digital signal processing (DSP) core, the DSP core performing signal and image processing and the OS core running an operating system and at least one user interface.

7. The ultrasound system of claim 1, wherein the single IC is based on a dual-core architecture comprising an operating system (OS) core and a digital signal processing (DSP) core, the DSP core performing signal and image processing and the OS core running an operating system and at least one user interface, the operating system being one of Linux and Windows.

8. The ultrasound system of claim 1, wherein the single IC is based on a dual-core architecture comprising first and second cores, the system further comprising a memory storing software accessible by the first and second cores.

9. The ultrasound system of claim 1, the single IC further comprising at least one communication interface for interfacing with at least one external device, the at least one communication interface being one of compact flash (CF), secure digital (SD), mini SD, USB, UART, and Ethernet.

10. A diagnostic medical imaging system, comprising:
a front-end portion for acquiring imaging data indicative of a patient;
a back-end portion comprising at least one multiple-core integrated circuit (IC), the multiple-core IC having at least first and second cores, the first core running an operating system and at least one user interface and the second core performing signal and image processing; and
a housing holding the front-end portion and the back-end portion there-within.

11. The system of claim 10, wherein the system is at least one of hand-carried, hand-held, and pocket-sized.

12. The system of claim 10, wherein the system is one of less than 500 grams in weight, less than 1 Kg in weight, and less than 3 Kg in weight.

13. The system of claim 10, wherein the system consumes one of under 5 Watts of system power and under 10 Watts of system power.

14. The system of claim 10, the housing further comprising at least one interface port for interfacing with at least one external component.

15. The system of claim 10, the front-end portion further comprising a field programmable gate array (FPGA) providing real-time control for acquiring the imaging data.

16. A method for acquiring and processing ultrasound data, comprising:
acquiring ultrasound data using a front-end of an ultrasound system;
running an operating system (OS) and user interface with a first core of a single integrated circuit (IC), the single IC being based on a multiple-core architecture comprising at least first and second cores; and
performing signal and image processing with the second core of the single IC.

17. The method of claim 16, further comprising outputting processed image data through a port provided on the single IC.

18. The method of claim 16, further comprising performing at least a portion of the signal and image processing with a third core of the single IC, the second and third cores processing at least one dynamically migrating application.

19. The method of claim 16, further comprising:
outputting processed image data through a video port provided on the single IC; and
displaying the processed image data on a display.

20. The method of claim 16, further comprising:
downloading digital signal processing applications to the single IC; and
storing the digital signal processing applications in a memory on the single IC.
